(19) United States
(12) Patent Application Publication
(10) Pub. No.: US 2005/0123583 A1
(43) Pub. Date: Jun. 9, 2005

(54) Medical use of reuterin

(76) Inventors: Hsing-Wen Sung, Hsinchu (TW); Chun-Nan Chen, Hsinchu (TW); Hsiang-Fa Liang, Taipei County (TW); Hosheng Tu, Newport Beach, CA (US)

Correspondence Address: HOSHENG TU, 15 RIEZ, NEWPORT BEACH, CA, 92657-0116, US

(21) Appl. No.: 10/924,538

(22) Filed: Aug. 24, 2004

Related U.S. Application Data
(63) Continuation-in-part of application No. 10/282,852, filed on Oct. 29, 2002.
Application No. 10/282,852 is a continuation-in-part of application No. 09/737,482, filed on Dec. 18, 2000, now abandoned.

(51) Int. Cl. A61K 38/16; A61M 31/00

(52) U.S. Cl. 424/426; 514/12; 604/500

(57) ABSTRACT

Use of reuterin, a naturally occurring β-hydroxypropinoaldehyde, in the manufacture of a biocompatible implant is disclosed, which involves crosslinking an amine-containing biological material such as chitosan, collagen, elastin, gelatin, fibrin glue, and combination thereof with reuterin.

### CROSS-REFERENCE TO RELATED APPLICATIONS

This application is a continuation-in-part of U.S. patent application Ser. No. 10/282,852, filed Oct. 29, 2002, which is a continuation-in-part of U.S. patent application Ser. No. 09/737,482, filed Dec. 18, 2000, now abandoned, the entireties of both are hereby incorporated by reference.

### FIELD OF THE INVENTION

The present invention is related to medical use of reuterin, and more particularly, to biological implants chemically treated with a naturally occurring reuterin.

### BACKGROUND OF THE INVENTION

Axelsson and co-workers reported the discovery of a broad-spectrum antimicrobial reagent termed reuterin (β-hydroxypropinoaldehyde) produced by Lactobacillus reuteri (Axelsson, L. et al., “Production of a broad spectrum antimicrobial substance by Lactobacillus reuteri,” Microb. ecoli., 2, 131-136, 1989). Lactobacillus reuteri resides in the gastrointestinal tract of humans and animals and is a naturally occurring substance. Cultures of Lactobacillus reuteri have been shown to accumulate large quantities of reuterin during anaerobic growth in the presence of glycerol (Axelsson, L. et al., “Production of a broad spectrum antimicrobial substance by Lactobacillus reuteri,” Microb. ecoli., 2, 131-136, 1989; Talarico, T. L. et al., “Production and isolation of reuterin, a growth inhibitor produced by Lactobacillus reuteri,” Antimicrob. agents chemother., 32, 1854-1858, 1988). Preliminary investigations indicate that it is a low-molecular-weight, neutral, water-soluble substance which has antibacterial, antimycotic, and antiprotozoal activity (Axelsson, L. et al., “Production of a broad spectrum antimicrobial substance by Lactobacillus reuteri,” Microb. ecoli., 2, 131-136, 1989).

Reuterin is a low cytotoxic substance that has been used in meat decontamination and preservation (El-ziney M. G. et al., “Application of reuterin produced by Lactobacillus reuteri 12002 for meat decontamination and preservation,” J of Food Protection, 61(3), 257-261, 1999). Reuterin has been fully characterized to be a naturally occurring low-toxicity fixative distinguishable itself from those more cytotoxic high-molecular weight aldehydes (El-ziney M. G. et al., “Characterization of growth and metabolite production of Lactobacillus reuteri during glucose/glycerol cofermentation in batch and continuous cultures,” Biotechnology Letter, 20 (10), 913-916, 1998; Yunmbam M. K. et al., “In vivo evaluation of reuterin and its combinations with suramin, melarsoprol, DL-α-difluoromethylomithine and bleomycin in mice infected with trypanosoma brucei brucei,” Comp. Biochem. Physiol. 105C(3), 521-524, 1993; Talarico T. L., “Chemical characterization of an antimicrobial substance produced by Lactobacillus reuteri,” Antimicrob. agents chemother., 33, 674-679, 1989; Yunmbam M. K. et al., “The in vitro efficacy of reuterin on the culture and bloodstream forms of trypanosoma brucei brucei,” Comp. Biochem. Physiol. 1051(2), 235-238, 1992).

Wolf et al. in U.S. Pat. No. 6,103,227, the entire contents of which are incorporated herein by reference, discloses a method of inhibiting the severity of Cryptosporidium parvum infection by enterally administering a therapeutically effective amount of Lactobacillus reuteri.

In one aspect, reuterin shows some characteristics of tissue fixation properties with extremely low cytotoxicity and might be used as a naturally occurring fixative. Various chemical fixatives including formaldehyde, glutaraldehyde, dialdehyde starch, and epoxy compounds have been used in fixing biological tissues; however, they all suffer from some degrees of cytotoxicity disadvantages. Clinically, the most commonly used fixative is glutaraldehyde (Nimni, M. E. et al., “Bioprosthesis derived from cross-linked and chemically modified collagenous tissues,” in Collagen Vol. III, M. E. Nimni (ed.), CRC Press, Boca Raton, Fla., 1988, pp. 1-38). Glutaraldehyde-fixed biological tissues have been used extensively to fabricate prosthetic heart valve prostheses, pericardial patches, vascular grafts, and ligament substitutes. However, the tendency for glutaraldehyde to markedly alter tissue stiffness, promote tissue calcification, and continuously leach cytotoxic residues are well recognized drawbacks of this chemical fixative.

To overcome the aforementioned deficiencies and disadvantages with the glutaraldehyde-fixed bioprostheses, the inventors of the present application developed a new fixation technique using genipin to fix biological tissues as disclosed in PCT WO 98/19718, wherein a biocompatible cross-linked material, suitable for use in implants, wound dressings, and blood substitutes was provided. The materials are prepared by crosslinking biological substances, such as collagen, chitosan, or hemoglobin, with genipin, a naturally occurring crosslinking agent. The crosslinking agent has much lower toxicity than conventionally used reagents, and the cross-linked products have good thermal and mechanical stability as well as biocompatibility. The PCT WO 98/19718 patent application, entitled “Chemical modification of biomedical materials with genipin”, is incorporated herein by reference.

Coury et al. in U.S. Pat. No. 6,162,241, the entire contents of which are incorporated herein by reference, discloses a hemostatic tissue sealant in which an amine containing hydrogel is crosslinked, wherein the tissue sealant may also comprise gelatin, collagen, albumin, ovalbumin and synthetic polyamino acids. Coury et al. further teaches that the crosslinking can be performed using an aldehyde. The majority of aldehydes are man-made chemicals which exhibit toxicity intolerable to tissue and cells. Nevertheless, Coury et al. does not teach the crosslinking process using a naturally occurring reuterin with antimicrobial property and extremely low cytotoxicity. Reuterin is distinguishable from other aldehydes in that it is a naturally occurring substance with characteristics of antibacterial, antimycotic, and antiprotozoal activities, and extremely low cytotoxicity.

Wallace et al. in U.S. Pat. No. 6,066,325, the entire contents of which are incroporated herein by reference, discloses a fragmented polymeric composition in which hydrogel is crosslinked, wherein the polymeric composition may also comprise chitosan, gelatin, collagen and hemoglobin. Wallace et al. '325 is distinguishable with a crosslinking process performed using an aldehyde. However, Wallace et al. does not teach the crosslinking process using a naturally occurring reuterin with antimicrobial property and extremely low cytotoxicity and with characteristics of antibacterial, antimycotic, and antiprotozoal activities.

Kerwin in U.S. Pat. No. 6,160,098, the entire contents of which are incorporated herein by reference, discloses a method for control of functionality during hemoglobin crosslinking with glutaraldehyde and glycoaldehyde, both are known chemicals with high cytotoxicity. There are disadvantages with the prior art including, in particular, the tendency for glutaraldehyde to markedly alter tissue stiffness and promote tissue calcification. However, Kerwin does not teach the crosslinking process using a naturally occurring reuterin with extremely low cytotoxicity and with characteristics of antibacterial, antimycotic, and antiprotozoal activities.

Walker in U.S. Pat. No. 4,060,677, the entire contents of which are incorporated herein by reference, discloses a method for regulating polymer molecular weight in the preparation of homopolymers, wherein various aldehyde reagents are cross-linked. Walker '677 is distinguishable with a crosslinking process performed using an aldehyde on synthetic polymer. However, Walker does not teach the crosslinking process for a biocompatible material useful for medical applications utilizing a naturally occurring reuterin with extremely low cytotoxicity and with characteristics of antibacterial, antimycotic, and antiprotozoal activities.

Yoshinaga in U.S. Pat. No. 5,276,088, the entire contents of which are incorporated herein by reference, discloses a method for synthesizing cyclodextrin polymers having amino and hydroxyl groups wherein polyvinyl alcohol is reacted with aldehydes. Yoshinaga '088 is distinguishable with a crosslinking process performed using an aldehyde on synthetic polymer. Further, Yoshinaga does not teach the crosslinking process for a biocompatible material useful for medical applications utilizing a naturally occurring reuterin with extremely low cytotoxicity and with characteristics of antibacterial, antimycotic, and antiprotozoal activities.

Sung et al. in U.S. Patent Application publication 2002/0122816, the entire contents of which are incorporated herein by reference, discloses the use of reuterin in the manufacture of a biocompatible implant, substitute or wound dressing by crosslinking with an amino-containing biological material. However, this patent application does not disclose a stent made of or comprised of reuterin treated biomaterial.

In another aspect, reuterin shows some characteristics of antibacterial, antimycotic, and antiprotozoal properties with extremely low cytotoxicity and might be used as a naturally occurring substance for mitigating vascular lesion or restenosis. Cultures of Lactobacillus reuteri have been shown to accumulate large quantities of reuterin during anaerobic growth in the presence of glycerol, wherein Lactobacillus reuteri resides in the gastrointestinal tract of humans and animals and is a naturally occurring substance. Restenosis is a common problem associated with angioplasty, particularly the percutaneous transluminal coronary angioplasty, and/or vascular stenting, among other procedures. Some aspects of the present invention relate to a cardiovascular stent having reuterin with dual functions: crosslinking the loaded biological material and providing antimicrobial, antibacterial, antimycotic, and antiprotozoal properties.

Restenosis of the blood vessel may develop over several months after the angioplasty or stenting procedures. Although stents are significant innovations in the treatment of stenosed vessels, there remains a need for administering therapeutic substances to the treatment site for mitigating restenosis. One major cause of restenosis is attributed to neointimal hyperplasia in response to tissue injury. To provide an efficacious concentration of the therapeutic substances to the treatment site, systemic administration often produces adverse or toxic side effects for the patients. Local delivery, such as loading the therapeutic substance onto the stent struts, is highly desirable with an effective dose of therapeutic substances for restenosis treatment with fewer side effects. The naturally occurring substances, such as genipin and reuterin, may be useful for effective restenosis therapy.

One technique for local delivery of therapeutic substances is through a polymer carrier coated onto the surface of a stent. A composition of one or more therapeutic substances with a proper carrier is formed so as for the therapeutic substance (for example, drug) to slowly leach out of the carrier matrix or elute out of the stent along with the biodegradable/bioerodible polymer or collagen carrier. A biocompatible collagen based carrier that is rendered less antigenic by reuterin is preferably in need.

There is, therefore, a clinical need for providing a crosslinking agent for biological tissues and/or a biomaterial with therapeutic effects for treating tissues having an improved performance in biocompatibility, cytotoxicity, and mechanical stability. The biomaterial preferably has additional characteristics of sterilization, and antibacterial, antimycotic, and antiprotozoal properties with extremely low cytotoxicity.

### SUMMARY OF THE INVENTION

It is one object of the present invention to provide an implant chemically treated with antimicrobial reuterin. In one embodiment, the implant is a stent that is either a durable stent or a biodegradable stent.

Some aspects of the invention relate to a method of treating a target tissue in a patient comprising administering a therapeutically effective amount of reuterin to the patient via an implant loaded with reuterin, wherein the effective amount of reuterin is characterized with extremely low cytotoxicity of MTT₅₀ about 20 ppm or higher.

In a further embodiment, the step of loading reuterin onto an implant is carried out with a carrier or medium onto the implant, wherein the carrier is a biodegradable polymer selected from a group consisting of polylactic acid (PLA), polyglycolic acid (PGA), poly (D,L-lactide-co-glycolide), polycaprolactone, and co-polymers thereof. In a further embodiment, the carrier is a biological material selected from a group consisting of collagen, gelatin, elastin, chitosan, fibrin glue, biological sealant, chitosan-alginate complex, and combination thereof.

In some aspects, the method of the present invention comprises contacting the biological material and reuterin in an aqueous medium at a temperature ranging from 4° C. to 50° C., preferably from 25° C. to 45° C., for a period ranging from 5 hours to 60 hours, preferably about 40 to 55 hours. In one embodiment, the aqueous medium has a concentration of reuterin ranging from 0.01 M to 1.0 M, and more preferably from 0.03 M to 0.2 M. In another embodiment, the aqueous medium has a pH value ranging from 3 to 12, and preferably from 4 to 10.5.

Some aspects of the invention relate to a method of treating a target tissue in a patient comprising administering a therapeutically effective amount of reuterin and a second therapeutically effective amount of at least one bioactive agent to the patient via an implant loaded with both the reuterin and the bioactive agent, wherein the bioactive agent is selected from a group consisting of actinomycin D, paclitaxel, vincristin, methotrexate, angiopeptin, batimastat, halofuginone, sirolimus, tacrolimus, everolimus, ABT-578, tranilast, dexamethasone, heparin, aspirin, mycophenylic acid, and the like.

Some aspects of the invention relate to a method of treating a target tissue in a patient comprising administering a therapeutically effective amount of a crosslinking agent to the patient via a stent loaded with the crosslinking agent, wherein the crosslinking agent is selected from a group consisting of reuterin, genipin, epoxy compounds, dialdehyde starch, glutaraldehyde, formaldehyde, dimethyl suberimidate, carbodiimides, succinimidyls, diisocyanates, and the like. In a further embodiment, the tissue to be treated is atherosclerotic tissue or vulnerable plaque.

### BRIEF DESCRIPTION OF THE DRAWINGS

Additional objects and features of the present disclosure will become more apparent and the disclosure itself will be best understood from the following Detailed Description of the Exemplary Embodiments, when read with reference to the accompanying drawings.

FIG. 1A shows optical density (O.D.) readings of the 3T3 fibroblasts cultured in the media added with varying concentrations of glutaraldehyde obtained in the MTT assay.

FIG. 1B shows optical density (O.D.) readings of the 3T3 fibroblasts cultured in the media added with varying concentrations of reuterin obtained in the MTT assay.

FIG. 2A shows fixation indices of the glutaraldehyde-fixed or reuterin-fixed tissues obtained at distinct elapsed fixation duration periods, wherein the rectangular dots represent the glutaraldehyde-fixed tissues and the round dots represent the reuterin-fixed tissues.

FIG. 2B shows denaturation temperatures of the glutaraldehyde-fixed or reuterin-fixed tissues obtained at distinct elapsed fixation duration periods.

FIG. 3A shows fixation indices of the tissues fixed by reuterin at different pHs.

FIG. 3B shows denaturation temperatures of the tissues fixed by reuterin at different pHs.

FIG. 4A shows fixation indices of the tissues fixed by reuterin at different temperatures.

FIG. 4B shows denaturation temperatures of the tissues fixed by reuterin at different temperatures.

FIG. 5A shows fixation indices of the tissues fixed by reuterin at different initial fixative concentrations.

FIG. 5B shows denaturation temperatures of the tissues fixed by reuterin at different initial fixative concentrations.

FIG. 6 shows chemical structures of reuterin and its derived forms. (a) Chemical structures of monomeric, hydrated monomeric, and cyclic dimeric forms of reuterin; (b) Other forms of oligomers of reuterin molecules represented by an addition of their hydroxyl group to aldehyde group.

FIG. 7 shows schematic illustration of the crosslinking structure of the reuterin-fixed tissue.

### DETAILED DESCRIPTION OF THE EXEMPLARY EMBODIMENTS

The preferred embodiments of the present invention described below relate particularly to a method of treating tissue in a patient comprising administering a therapeutically effective amount of naturally occurring fixative, such as reuterin or genipin, to the patient via a stent loaded, impregnated, or coated with reuterin and/or genipin. While the description sets forth various embodiment specific details, it will be appreciated that the description is illustrative only and should not be construed in any way as limiting the invention. Furthermore, various applications of the invention, and modifications thereto, which may occur to those who are skilled in the art, are also encompassed by the general concepts described below.

Reuterin, as a naturally occurring substance, has antibacterial, antimycotic, and antiprotozoal activities as described in the articles mentioned above. Additionally, it is discovered that reuterin, β-hydroxypropinoaldehyde, can react with free amino groups within biological tissues. Therefore in some aspects, reuterin can be used as a crosslinker (fixative) and a sterilant for biological tissues, natural products, or synthetic polymers in clinical applications.

Reuterin has the following general chemical structure:
HO—CH₂—CH₂—CH═O
and can be produced by Lactobacillus reuteri under control conditions. Reuterin used in the following examples is identified by high performance liquid chromatography (HPLC). Lactobacillus reuteri resides in the gastrointestinal tract of humans and animals and is a naturally occurring substance, which is distinguishable from other cytotoxic synthetic aldehydes. Cultures of Lactobacillus reuteri have been shown to accumulate large quantities of reuterin during anaerobic growth in the presence of glycerol.

Antimicrobial activity of reuterin was studied (J Biomed Mater Res 2002;61:360-369) and disclosed in the present invention, wherein glutaraldehyde is used as a control. The microorganisms tested in the study are Escherichia coli (ATCC 25922), Pseudomonas aeruginosa (ATCC 27853), Staphylococcus aureus (ATCC 25923), and Bacillus subtillis (ATCC 6633). The results show that all tested microorganisms, including both the gram-positive bacteria (Staphylococcus aureus and Bacillus subtillis) and gram-negative bacteria (Escherichia coli and Pseudomonas aeruginosa), were sensitive to reuterin. Generally, 20 to 35 ppm of reuterin can prevent the growth of the tested microorganisms, while 40 to 50 ppm of reuterin resulted in the death of the tested microorganisms. However, the values for glutaraldehyde were significantly greater than those for reuterin (approximately 2˜3 times higher). This indicated that the antimicrobial activity of reuterin is significantly superior to glutaraldehyde.

The cytotoxicity of reuterin was also studied (J Biomed Mater Res 2002;61:360-369) and disclosed in the present invention, wherein glutaraldehyde is again used as a control. The cytotoxicity of the test reagents (glutaraldehyde vs. reuterin) was evaluated in vitro using a mouse-derived established cell line of 3T3 fibroblasts (BALB/3T3 C1A31-1-1). The assay (light microscopic observation and MTT assay) was used to measure the proportion of viable cells following a test reagent-treated culture.

In the assay, 3T3 fibroblasts were seeded in 24-well plates at 5×10⁴ cells/well in 1 ml Dulbecco's modified eagle medium (DMEM, Gibco 430-2800EG, Grand Island, N.Y., USA) with 10% fetal calf serum (FCS, Hyclone Laboratories, Logan, Ut., USA). The cell culture was maintained in a humidified incubator at 37° C. with 10% CO₂ in air. Cells in log phase of growth were then exposed to a new DMEM medium drugged with varying concentrations of glutaraldehyde or reuterin. After 24 hours of culture, the growth media in the wells were removed and the cells were photographed using light microscopy. Subsequently, the cells were washed with phosphate buffered saline (PBS) twice and surviving cell numbers were then determined indirectly by 3-(4,5-dimethylthiazol-yl)-2,5-diphenyltetrazolium bromide (MTT, Sigma Chemical Co., St. Louis, Mo., USA) dye reduction.

The MTT assay is based on the reduction of MTT, a yellow soluble dye by the mitochondrial succinate dehydrogenase to form an insoluble dark blue formazan product. Only viable cells with active mitochondria reduce significant amounts of MTT to formazan. In the test, 200 μl MTT solution (0.5 g/l in medium, filter-sterilized) was added to the culture wells. After incubation for 3 hours at 37° C. in a 10% CO₂ atmosphere, the MTT reaction medium was removed and blue formazan was solubilized by 100 μl dimethylsulfoxide (DMSO). Optical density readings were then performed using a multiwell scanning spectrophotometer (MRX Microplate Reader, Dynatech Laboratories Inc., Chantilly, Va., USA) at a wavelength of 570 nm.

A photomicrograph of the 3T3 fibroblasts cultured in the medium without any test crosslinking reagent showed that the cells cultured in the control medium were confluent, which is used as our control in the evaluation of the cytotoxicity of glutaraldehyde and reuterin. Photomicrographs of the 3T3 fibroblasts cultured in the media treated with varying concentrations of glutaraldehyde or reuterin revealed that: (a) the cells cultured in the medium treated with an extremely low concentration of glutaraldehyde (0.05 ppm) were confluent; (b) as the concentration of glutaraldehyde increased to 5 ppm, all the cells cultured were found dead; and (c) in contrast, as the concentration of reuterin increased to 15 ppm, the cells cultured were confluent.

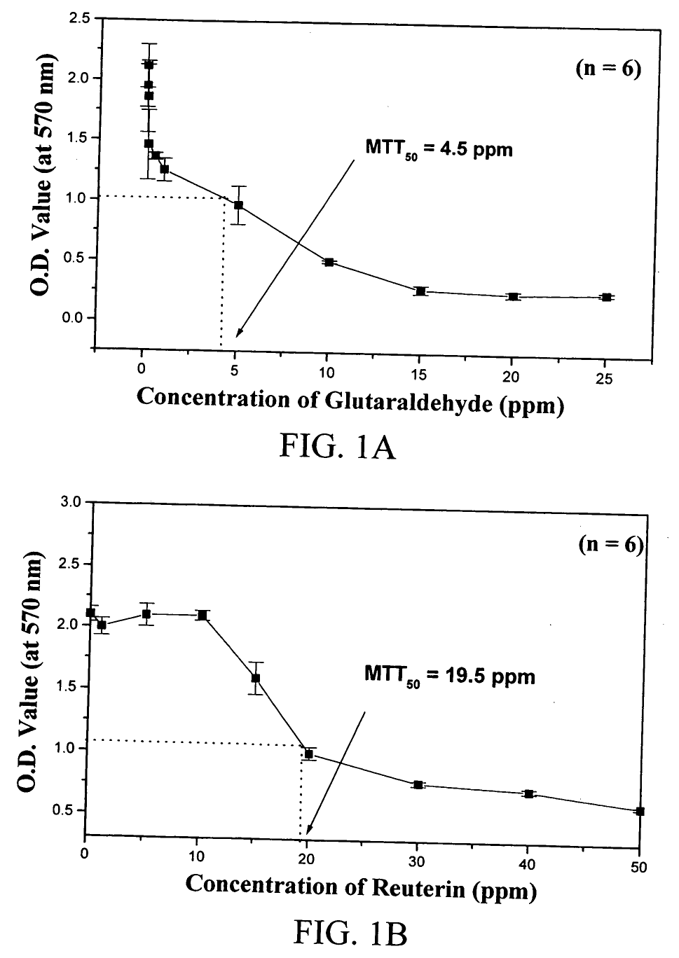

FIGS. 1A and 1B illustrate the optical density readings of the 3T3 fibroblasts cultured in the media drugged with varying concentrations of glutaraldehyde or reuterin obtained in the MTT assay. As shown in the figures, the optical density reading of the cells cultured in the medium drugged with glutaraldehyde declined more remarkably than that drugged with reuterin, as the concentration of the test reagent increased. The MTT₅₀ concentration was determined as the concentration of the test reagent required to reduce the optical density reading to half that of the control.

The MTT₅₀ concentration of glutaraldehyde was approximately 4 ppm, which was much lower than that of reuterin about 20 ppm. It is one aspect of the present invention to provide a biocompatible material useful for medical applications comprising a chemically treated biological material formed by treating an amine-containing biological material with reuterin, wherein reuterin is characterized with extremely low toxicity of MTT₅₀ about 20 ppm or higher. The biocompatible material may be selected from a group consisting of an implant, drug carrier, substitute, or wound dressing.

### EXAMPLE 1

### Fixation of Biological Tissues

Materials and Methods: In this example, fresh porcine pericardia procured from a slaughterhouse are used as raw materials. The procured pericardia were transported in a cold physiological saline solution. Upon received, the pericardia were first gently rinsed with fresh saline to remove excess blood on the tissue. Adherent fat then was carefully trimmed from the pericardial surface. The maximum period between retrieval and initiation of tissue fixation was consistently less than 6 hours.

In the first part of this example, the rate of tissue fixation by reuterin was investigated. Glutaraldehyde was used as a control. The trimmed pericardia were first fixed in a 0.068M aqueous glutaraldehyde or reuterin solution buffered with phosphate-buffered saline (PBS, pH 7.4) at room temperature (25° C.). The amount of solution used in each fixation was approximately 100 mL for each 6×6 cm porcine pericardium. Samples of each studied group then were taken out at distinct elapsed fixation duration periods (at 5 min, 1 h, 4 h, 12 h, 24 h, 48 h, and 72 h after the initiation of tissue fixation, respectively). The rate of tissue fixation by reuterin was determined by monitoring the changes in fixation index and/or denaturation temperature of the fixed tissues during the course of fixation.

In the second part of this example, the effects of fixation conditions (pH, temperature, and initial fixative concentration) on the degrees of tissue fixation by reuterin were investigated. The degree of tissue fixation by reuterin was determined by measuring the crosslinking characteristics (fixation index and denaturation temperature) of the fixed tissue. To elucidate the effects of pH on the degree of tissue fixation by reuterin, a 0.068M aqueous reuterin solution was buffered with: citric acid/sodium citrate (pH 4.0); PBS (pH 7.4); sodium borate (pH 8.5); or sodium carbonate/sodium bicarbonate (pH 10.5) at room temperature (25° C.). The effects of temperature on the degree of tissue fixation by reuterin were evaluated at: 4° C., 25° C., 37° C., or 45° C. A 0.068M aqueous reuterin solution buffered at pH 7.4 was used. To elucidate the effects of initial fixative concentration on the degree of tissue fixation by reuterin, a 0.034M, 0.068M, 0.1M, or 0.2M aqueous reuterin solution buffered at pH 7.4 at 25° C. was used. The duration for each fixation was 72 h.

The fixation index, determined by the ninhydrin assay, was defined as the percentage of free amino groups in tissue reacted with the test crosslinking agent subsequent to fixation. In the ninhydrin assay, the test tissue was first lyophilized for 24 hours and then weighed. Subsequently, the lyophilized tissue was heated with a ninhydrin solution for 20 min. After heating with ninhydrin, the optical absorbance of the solution was recorded with a spectrophotometer (Model UV-150-O₂, Shimadzu Corp., Kyoto, Japan) using glycine at various known concentrations as standard. It is known that the amount of free amino groups in the test tissue, after heating with ninhydrin, is proportional to the optical absorbance of the solution.

The denaturation temperature of each studied group was measured in a Perkin-Elmer differential scanning calorimeter (Model DSC 7, Norwalk, Conn.). This technique has been widely used in studying the thermal transitions of collagenous tissues.

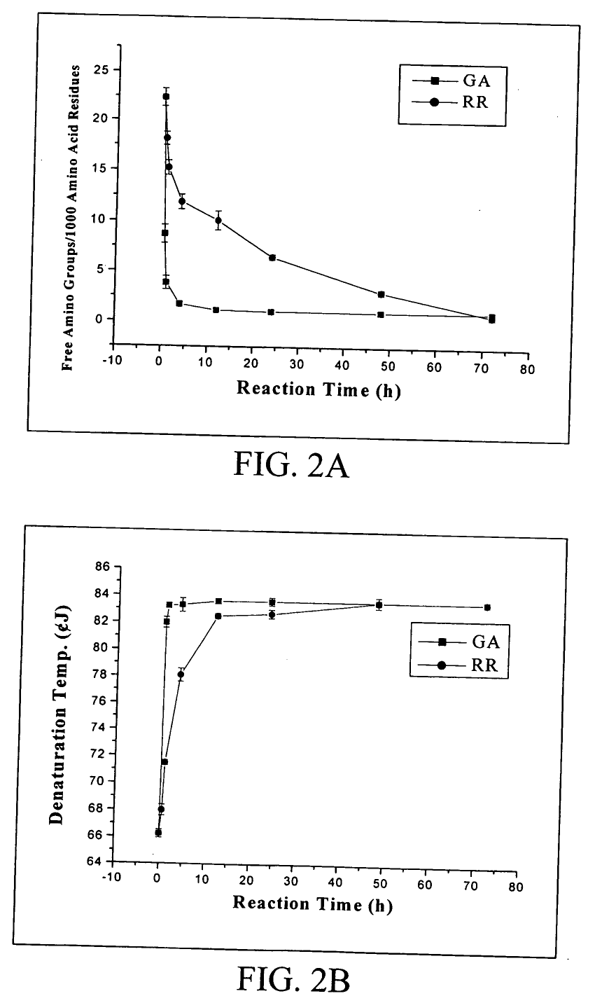

FIGS. 2A and 2B compare the fixation indices and denaturation temperatures of the tissues fixed with glutaraldehyde or reuterin obtained at various elapsed fixation duration periods. As shown in FIG. 2A and FIG. 2B, both the fixation index and denaturation temperature of the glutaraldehyde-fixed tissue increased more rapidly than the reuterin-fixed tissue at the beginning of fixation. However, after 48 hours of fixation, the fixation index and denaturation temperature of both studied groups were comparable.

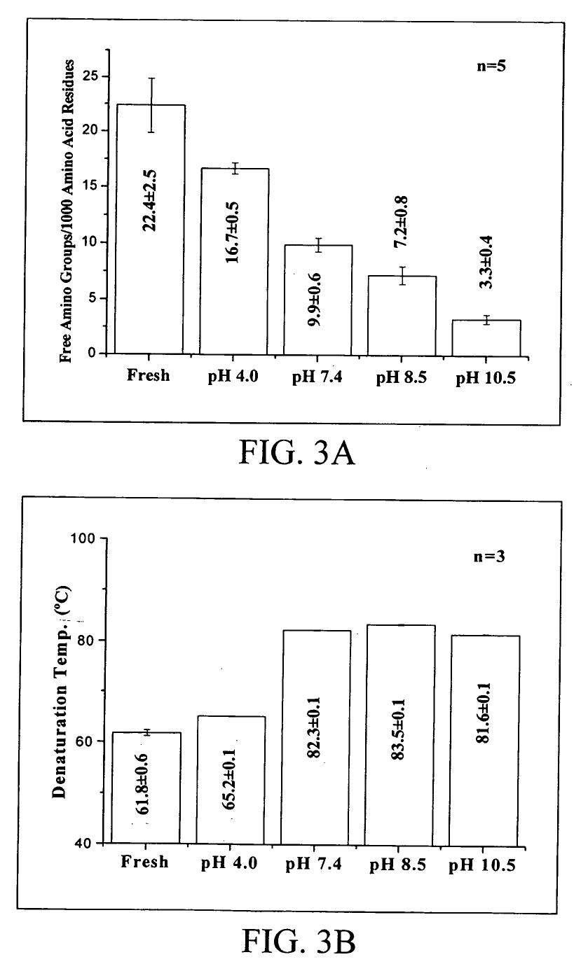

The pH of the buffer used in fixation played an important role in affecting the crosslinking characteristics of the reuterin-fixed tissue. FIGS. 3A and 3B present the fixation indices and denaturation temperatures of the tissues fixed by reuterin under various pHs. In general, the fixation indices of the reuterin-fixed tissues increased with increasing the fixation pH value. The denaturation temperatures of the tissues fixed by reuterin at pH 7.4 or pH 8.5 were relatively greater than that fixed at pH 10.5, while the tissue fixed at pH 4.0 had the lowest fixation indices and the lowest denaturation temperature.

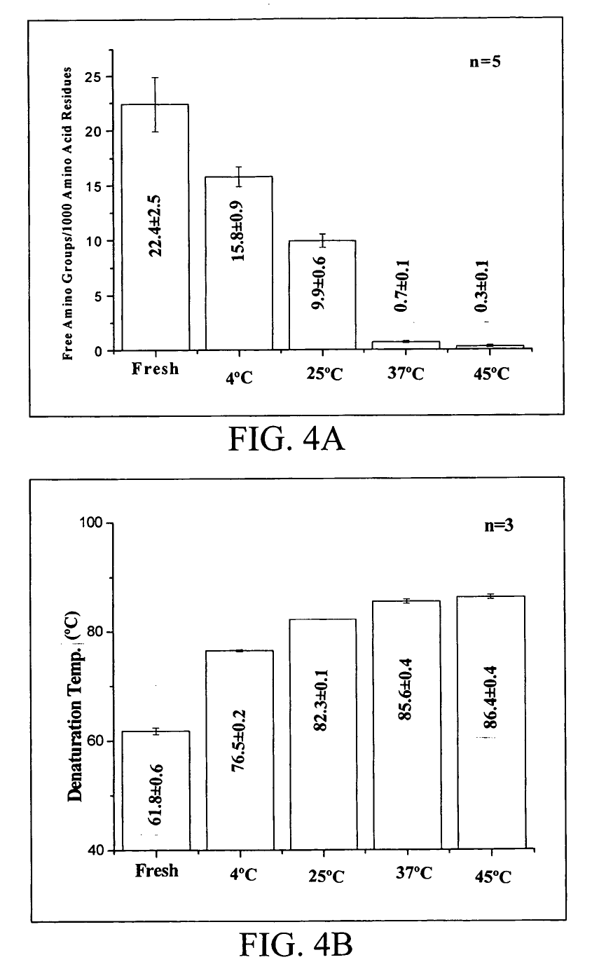

The fixation temperature significantly influenced the crosslinking characteristics of the reuterin-fixed tissue. The effects of temperature on the fixation index and denaturation temperature of the reuterin-fixed tissue are presented in FIGS. 4A and 4B. As indicated in FIG. 4A and FIG. 4B, the tissues fixed at 37° C., or 45° C. had comparable fixation indices and denaturation temperatures. In contrast, the tissue fixed at 4° C. had the lowest fixation index and the lowest denaturation temperature among all groups studied at different temperatures.

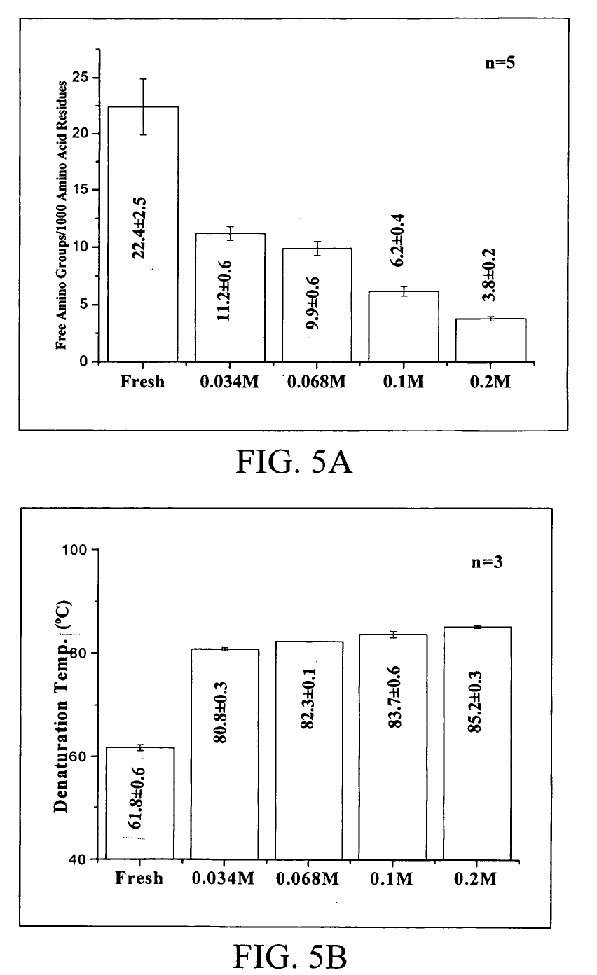

The effects of initial fixative concentration on the crosslinking characteristics of the reuterin-fixed tissue are given in FIGS. 5A and 5B. As given in FIG. 5A and FIG. 5B, the fixation indices increased with increased initial fixative concentrations and denaturation temperatures of the tissues fixed by reuterin at different initial fixative concentrations were approximately equivalent.

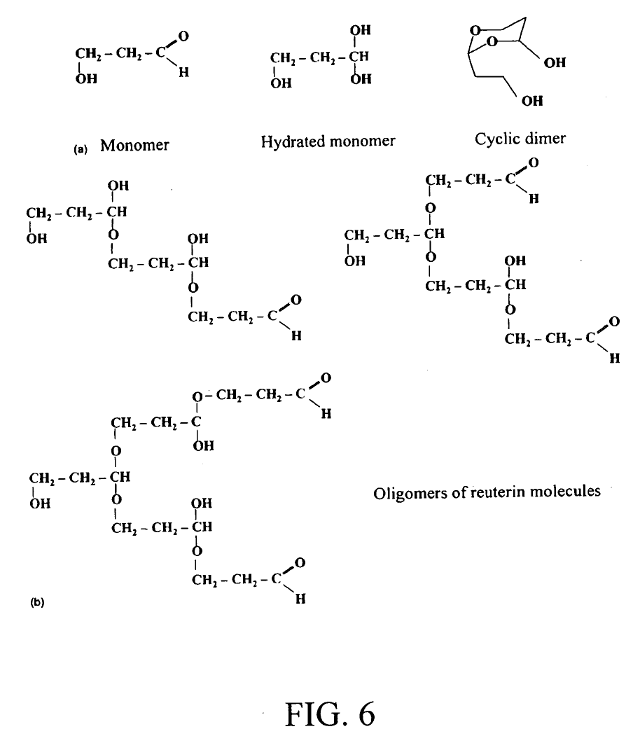

It was reported that aqueous reuterin is an equilibrium mixture of monomeric, hydrated monomeric, and cyclic dimeric forms of β-hydroxypropinoaldehyde. FIG. 6 shows chemical structures of reuterin and its derived forms. (a) Chemical structures of monomeric, hydrated monomeric, and cyclic dimeric forms of reuterin; (b) Other forms of oligomers of reuterin molecules represented by an addition of their hydroxyl group to aldehyde group (Chen C N et al., J Biomed Mater Res 2002;61:360-369). Some aspects of the present invention relate to a biological stent (biodegradable and non-biodegradable) having reuterin with dual functions: crosslinking the loaded biological material and providing antimicrobial, antibacterial, antimycotic, and antiprotozoal properties.

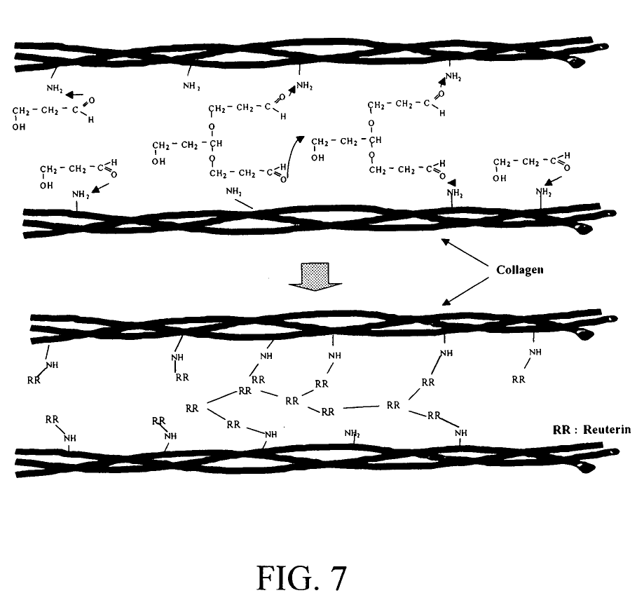

FIG. 7 shows a proposed schematic illustration of the crosslinking structure of the reuterin-fixed tissue. The degree of tissue fixation by a crosslinking agent may depend on the fixation conditions (pH, temperature, and fixative concentration) at which the reaction takes place. As shown, the reaction of reuterin with biological tissue requires a nucleophilic agent, the non-protonated free amino groups of lysine, hydroxylysine, or arginine residues in tissue. The ionization equilibrium of the free amino groups is given by
R—NH₃⁺ [symbol] R—\overline{N}H₂+H⁺

To increase the reaction of reuterin with tissue, it is required to convert the free amino groups in tissue into a more strongly nucleophilic agent or to increase the molar proportion of non-protonated to protonated free amino groups. Therefore, it is suggested that a larger denaturation temperature and mechanical strength, and a better resistance against enzymatic degradation of the fixed tissue are expected under a higher fixation pH (Sung H W et al. Biomaterials 2003;24:1335-1347).

### EXAMPLE 2

### Biocompatibility Study and Subcutaneous Study

To evaluate the biocompatibility of the biological tissues fixed with reuterin, a subcutaneous study was conducted using a growing rat model. Fresh and the glutaraldehyde-fixed counterparts were used as controls.

Materials and Methods: Fresh porcine pericardia was used as raw materials and treated as in Example 1.

The trimmed pericardia were fixed in a 0.068M glutaraldehyde or reuterin solution at 37° C. for 3 days. The amount of solution used in each fixation was approximately 200 mL for a 6×6 cm² porcine pericardium. The reuterin solution was buffered with sodium borate (pH 8.5), whereas the glutaraldehyde solutions were buffered with phosphate buffered saline (0.01M, pH 7.4). After fixation the test samples were divided into two groups. For the first group, the fixed pericardia were rinsed in sterilized phosphate buffered saline with a solution change for several times for approximately 5 hours. For the second group, the fixed pericardia were sterilized with a series of ethanol solutions in an order of increasing concentration (20˜75%) for approximately 5 hours.

Subsequently, the test samples were implanted subcutaneously in a growing rat model (6-week-old male Wistar) under aseptic conditions. The implanted samples were retrieved at 3 days and 1, 4, and 12 weeks following the procedures. The denaturation temperatures of the retrieved samples were determined by a differential scanning calorimeter (Perkin Elmer Model DSC 7, Norwalk, Conn., USA). The content of calcium deposited on each retrieved sample was assessed with atomic absorption spectroscopy.

Results: In the gross examination, it was found that fresh samples were thinner than the other fixed samples at 1-week post implantation. At 4-week postoperatively, fresh samples were completely biodegraded, while the other fixed samples remained intact.

It was found that the denaturation temperatures of the same study group retrieved at different post implantation times were substantially the same. Of the fixed samples, the denaturation temperatures of the reuterin-fixed samples were comparable to their glutaraldehyde-fixed counterparts. The denaturation temperatures of the fixed samples were about 85° C., which was significantly greater than that (62° C.) of the fresh one.

The photomicrographs of the fresh, glutaraldehyde- and reuterin-fixed tissues stained with H&E retrieved at 3-day postoperatively showed that the fresh tissue had the most notable inflammatory reaction among all the study groups. The degrees in inflammatory reaction observed for the glutaraldehyde- and reuterin-fixed tissues retrieved at this time were not significantly different. At 4-week postoperatively, the degree of inflammatory reaction for each study group was more remarkable than its corresponding counterpart retrieved at 3-day postoperatively. As observed at 3-day postoperatively, the degrees in inflammatory reaction for the glutaraldehyde- and reuterin-fixed tissues were not significantly different.

The photomicrographs of the glutaraldehyde-, and reuterin-fixed tissues retrieved at 12-week postoperatively were also taken. It should be noted that no photomicrograph of the fresh tissue retrieved at this time could be made, due to its complete degradation. As observed in the photomicrographs, the degrees in inflammatory reaction for all the fixed samples were less notable than those retrieved at 1- and 4-week postoperatively. Of note is that the inflammatory cells surrounding the reuterin-fixed tissue were less than the glutaraldehyde-fixed tissue.

The results of the calcium contents for the fresh, glutaraldehyde-, and reuterin-fixed tissues before implantation and those retrieved at 3-day, 1-, and 4-week postoperatively are presented in Table I below. It should be noted that no data could be obtained for the fresh tissues retrieved at 4-week postoperatively, due to their complete disintegration. As presented in the table, the difference in calcium content between the samples before implantation and those retrieved at distinct implantation duration were not significant for all the study groups.

TABLE I
Calcium Contents (μg calcium/mg dry tissue weight)* of Each
Study Group Before Implantation and Retrieved at Distinct
Implantation Duration

| Implantation Duration | | Fresh | Glutaraldehyde | Reuterin |
|---|---|---|---|---|
| 0 | (n = 4) | 1.2 ± 0.1 | 1.4 ± 0.1 | 1.5 ± 0.3 |
| 3-day | (n = 4) | 1.3 ± 0.1 | 1.5 ± 0.3 | 1.5 ± 0.2 |
| 1-week | (n = 4) | 1.9 ± 0.2 | 2.1 ± 0.9 | 1.6 ± 0.3 |
| 4-week | (n = 4) | N/A^{#} | 1.8 ± 0.6 | 1.7 ± 0.5 |

*The numbers are presented in mean ± standard deviation.
^{#}N/A: Data were not available, due to complete degradation of the fresh tissues at 4-week postoperatively.

Additionally, the tensile strength of each retrieved sample was measured by an Instron Universal Testing Machine (Model 4302) at a constant speed of 50 mm/min. The results showed the tensile strengths of the reuterin-fixed and glutaraldehyde-fixed samples were comparable before implantation and retrieved at distinct duration periods postoperatively.

In another aspect, it is one object of the present invention to provide a method of treating tissue in a patient comprising administering a therapeutically effective amount of reuterin to the patient via an implant or a stent loaded with reuterin, wherein the effective amount of reuterin is characterized with extremely low cytotoxicity of MTT₅₀ about 20 ppm or higher. In one embodiment, the tissue to be treated is restenosis tissue or vulnerable plaque. Reuterin shows some characteristics of tissue fixation properties with extremely low cytotoxicity and might be used as a naturally occurring fixative. In one embodiment, the stent is a biodegradable stent made of biodegradable material or a durable stent made of a durable metal or plastic.

The step of loading may be carried out with a polymer carrier or a biological carrier admixed with reuterin onto the stent, wherein the polymer carrier may comprise biodegradable polymer, bioerodible polymer, shape memory polymer, collagen, elastin, chitosan, gelatin and the like. The biodegradable polymer may be selected from a group consisting of polylactic acid (PLA), polyglycolic acid (PGA), poly (D,L-lactide-co-glycolide), polycaprolactone, and co-polymers thereof. Though the mechanism of tissue treatment with reuterin is unclear, it is believed that reuterin slowly diffuses out of the implanted stent into the surrounding tissue so as to chemically treating and passivating the target tissue or lesion. From prior data, a chemically treated or fixed tissue, such as a bioprosthetic tissue valve with a crosslinking agent, becomes denatured with less antigenicity and less reactivity. It is believed that reuterin may react with the tissue lesion to render the lesion less antigenic and less reactive.

Reuterin is a low-molecular-weight, naturally occurring, water-soluble substance which has antibacterial, antimycotic, and antiprotozoal activities. It is also believed that reuterin slowly diffuses out of the implanted stent into the surrounding tissue so as to biochemically treating the target tissue or lesion. The therapeutic effect on treating tissue may be enhanced in a combined therapy as discussed below.

Genipin is a naturally occurring crosslinking agent with extremely low cytotoxicity, it is believed that genipin could be loaded onto the stent surface with a carrier (a polymer carrier or a biological carrier). The genipin slowly diffuses out of the implanted stent into the surrounding tissue to chemically treating and passivating the target tissue or lesion. It is one object of the present invention to provide a method of treating tissue in a patient comprising administering a therapeutically effective amount of naturally occurring fixative, for example genipin, to the patient via a stent loaded with genipin in a polymer carrier. In one embodiment, the tissue to be treated is restenosis tissue or vulnerable plaque.

Combined Therapy

Vascular injury causing intimal thickening can be broadly categorized as being either biologically or mechanically induced. Atherosclerosis is one of the most commonly occurring forms of biologically mediated vascular injury leading to stenosis. The migration and proliferation of vascular smooth muscle plays a crucial role in the pathogenesis of atherosclerosis. Atherosclerotic lesions include massive accumulation of lipid laden “foam cells” derived from monocyte/macrophage and smooth muscle cells. Formation of “foam cell” regions is associated with a breech of endothelial integrity and basal lamina destruction. Triggered by these events, stenosis is produced by a rapid and selective proliferation of vascular smooth muscle cells with increased new basal lamina (extracellular matrix) formation and results in eventual blocking of arterial pathways. As disclosed above, reuterin and/or genipin, a naturally occurring fixative with extremely low cytotoxicity, may be effective to partially denature and passivate the extracellular matrix of the lesion to mitigate the proliferation of vascular smooth muscle.

Mechanical injuries leading to intimal thickening result following balloon angioplasty, stenting, vascular surgery, transplantation surgery, and other similar invasive processes that disrupt vascular integrity. Intimal thickening following balloon catheter injury has been studied in animals as a model for arterial restenosis that occurs in human patients following balloon angioplasty. Injury is followed by a proliferation of the medial smooth muscle cells, after which many of them migrate into the intima through fenestrae in the internal elastic lamina and proliferate to form a neointimal lesion. As disclosed above, reuterin and/or genipin may be effective to partially denature the thicken tissue and mitigate the neointimal lesion.

Morris in U.S. Pat. No. 5,516,781, the entire contents of which are incorporated herein by reference, discloses a method of preventing or treating hyperproliferative vascular disease in a mammal in need thereof by administering an antiproliferative effective amount of rapamycin to the mammal orally or via a vascular stent loaded with rapamycin. Wright et al. in U.S. Pat. No. 6,273,913, the entire contents of which are incorporated herein by reference, discloses means for delivery of rapamycin from an intravascular stent mixed or bound to a polymer coating applied on stent to inhibit neointimal tissue proliferation and thereby prevent restenosis. It is one object of the present invention to provide a method of treating tissue in a patient comprising administering a therapeutically effective amount of crosslinking agent (such as reuterin or genipin) in combination with a therapeutically effective amount of rapamycin to the patient via a stent loaded or coated with the crosslinking agent.

Moses at the 2002 TCT meeting in Washington, D.C. presented the clinical and angiographic outcomes of a clinical study SIRIUS (Medscape Today viewarticle/442503, Oct. 11, 2002), the entire contents of which are incorporated herein by reference. Conducted in the United States, the SIRIUS (Sirolimus-eluting stent in de novo native coronary lesions) trial was a multicenter, randomized, double-blind, controlled study designed to evaluate the safety and efficacy of the sirolimus-eluting stent in reducing target vessel failure. Nine month follow-up results indicate that sirolimus-coated (rapamycin) stents significantly reduce the rates of in-stent and in-segment restenosis, target lesion revascularization, and neointimal hyperplasia in the distal margin of the stent. It is one object of the present invention to provide a method of treating tissue in a patient comprising administering a therapeutically effective amount of reuterin and a second therapeutically effective amount of rapamycin, in combination, to the patient via a stent loaded with both reuterin and rapamycin.

Therapeutic agents to inhibit restenosis have been used with varying success. Taxol, an antimicrotubule agent isolated from the bark of the Pacific Yew tree, is especially effective in inhibiting some cancers and is believed to be effective in combating restenosis. Zhong in U.S. Pat. No. 6,231,600, the entire contents of which are incorporated herein by reference, discloses stents with hybrid coating for medical devices. Zhong discloses the use of a polyfunctional aziridine as a crosslinking agent covalently bond to the polymer and the heparin. However, Zhong does not teach the incorporation of a naturally occurring fixative for crosslinking the tissue lesion upon released from the stent implant.

Colombo at the 2002 TCT meeting in Washington, D.C. presented the clinical outcomes of a clinical study TAXUS with slow- and moderate-release taxanes formulation (Medscape Today viewarticle/442693, Oct. 11, 2002), the entire contents of which are incorporated herein by reference. The TAXUS program is a series of clinical studies designed to collect data on Boston Scientific paclitaxel-eluting stents for the reduction of intracoronary neointimal tissue formation after angioplasty and stenting. The findings from the TAXUS study demonstrates the effectiveness of controlled release of paclitaxel for the treatment of de novo lesions as compared with bare metal stents. It is one object of the present invention to provide a method of treating tissue in a patient comprising administering a therapeutically effective amount of reuterin and a second therapeutically effective amount of bioactive agent, in combination, to the patient via a stent loaded with both reuterin and at least one bioactive agent.

In a co-pending application, U.S. patent application Ser. No. 10/916,170 filed Aug. 11, 2004, entitled “DRUG-ELUTING BIODEGRADABLE STENT”, the entire contents of which are incorporated herein by reference, it is disclosed that the bioactive agent may be selected from a group consisting of, but not limited to, actinomycin D, paclitaxel, vincristin, methotrexate, and angiopeptin, batimastat, halofuginone, sirolimus, tacrolimus, everolimus, ABT-578, tranilast, dexamethasone, heparin, aspirin, and mycophenylic acid. The application also discloses the carrier for bioactive agents that may be selected from a group consisting of collagen, gelatin, elastin, chitosan, fibrin glue, biological sealant, chitosan-alginate complex, and combination thereof.

Some aspects of the invention relate to a method of treating a target tissue in a patient comprising administering a therapeutically effective amount of a crosslinking agent to the patient via a stent loaded with the crosslinking agent, wherein the tissue to be treated is atherosclerotic tissue, vulnerable plaque, or the like. In a further embodiment, the crosslinking agent is a naturally occurring reuterin. In a further embodiment, the crosslinking agent is selected from a group consisting of genipin, epoxy compounds, dialdehyde starch, glutaraldehyde, formaldehyde, dimethyl suberimidate, carbodiimides, succinimidyls, diisocyanates, and the like.

Although the present invention has been described with reference to specific details of certain embodiments thereof, it is not intended that such details should be regarded as limitations upon the scope of the invention except as and to the extent that they are included in the accompanying claims. Many modifications and variations are possible in light of the above disclosure.

1. A method of treating a target tissue in a patient comprising administering a therapeutically effective amount of reuterin to said patient via an implant loaded with reuterin.

2. The method of claim 1, wherein the effective amount of reuterin is characterized with extremely low cytotoxicity of MTT₅₀ about 20 ppm or higher.

3. The method of claim 1, wherein the tissue to be treated is atherosclerotic tissue or vulnerable plaque.

4. The method of claim 1, wherein the implant is a stent.

5. The method of claim 4, wherein the stent is a durable stent or a biodegradable stent.

6. The method of claim 1, wherein the reuterin is a naturally occurring substance that is derived from Lactobacillus reuteri.

7. The method of claim 1, wherein the loading step is carried out with a carrier onto said implant.

8. The method of claim 7, wherein the carrier is a biodegradable polymer.

9. The method of claim 8, wherein the biodegradable polymer is selected from a group consisting of polylactic acid (PLA), polyglycolic acid (PGA), poly (D,L-lactide-co-glycolide), polycaprolactone, and co-polymers thereof.

10. The method of claim 7, wherein the carrier is selected from a group consisting of collagen, gelatin, elastin, chitosan, fibrin glue, biological sealant, chitosan-alginate complex, and combination thereof.

11. The method of claim 1 further comprising administering a second therapeutically effective amount of at least one bioactive agent, in combination with the reuterin, to said patient via the implant loaded with both the reuterin and the at least one bioactive agent.

12. The method of claim 11, wherein the at least one bioactive agent is selected from a group consisting of actinomycin D, paclitaxel, vincristin, methotrexate, angiopeptin, batimastat, halofuginone, sirolimus, tacrolimus, everolimus, ABT-578, tranilast, dexamethasone, heparin, aspirin, and mycophenylic acid.

13. A method of treating a target tissue in a patient comprising administering a therapeutically effective amount of a crosslinking agent to said patient via a stent loaded with said crosslinking agent.

14. The method of claim 13, wherein the tissue to be treated is atherosclerotic tissue or vulnerable plaque.

15. The method of claim 13, wherein the stent is a durable stent or a biodegradable stent.

16. The method of claim 13, wherein the crosslinking agent is a naturally occurring reuterin.

17. The method of claim 13, wherein the crosslinking agent is selected from a group consisting of genipin, epoxy compounds, dialdehyde starch, glutaraldehyde, formaldehyde, dimethyl suberimidate, carbodiimides, succinimidyls, and diisocyanates.

18. The method of claim 13, wherein the loading step is carried out with a carrier onto said stent, wherein the carrier is a biodegradable polymer.

19. The method of claim 18, wherein the biodegradable polymer is selected from a group consisting of polylactic acid (PLA), polyglycolic acid (PGA), poly (D,L-lactide-co-glycolide), polycaprolactone, and co-polymers thereof.

20. The method of claim 13, wherein the carrier is selected from a group consisting of chitosan, collagen, elastin, gelatin, fibrin glue, and combination thereof.
